(19) United States
(12) Patent Application Publication
(10) Pub. No.: US 2015/0018573 A1
(43) Pub. Date: Jan. 15, 2015

(54) Process for producing renewable bioproducts from various feedstocks

(71) Applicants: Zackery Allen McMurry, Mishawaka, IN (US); James Casey Smoot, West Sacramento, CA (US)

(72) Inventors: Zackery Allen McMurry, Mishawaka, IN (US); James Casey Smoot, West Sacramento, CA (US)

(21) Appl. No.: 13/987,228

(22) Filed: Jul. 13, 2013

(51) Int. Cl. C07C 67/02

(52) U.S. Cl. CPC C07C 67/02
USPC 560/187; 560/217

(57) ABSTRACT

The present invention describes a method for production of a class of renewable specialty chemicals that may be used for a variety of purposes including as a solvent in the semiconductor industry and as a reagent for commercial chemical production. The precursor compound, PHA, can be produced using various feedstocks such as waste water treatment plant biosolids, distiller's grains and agricultural residual biomass (e.g., corn stover and rice straw). The production of specialty chemicals in tandem with energy production can serve to aid the overall economics of renewable feedstock energy production.

### CROSS REFERENCE TO RELATED APPLICATIONS

This patent application claims priority to provisional patent application Ser. No. 61/741,664 for Use Of Alkoxyalkanoate Alchol Esters As Specialty Chemicals filed by Zackery Allen McMurry and James Casey Smoot on Jul. 25, 2012.

### BACKGROUND OF INVENTION

This present invention relates generally to the field of biorefinery production and more particularly the conversion of cellular biomass to renewable bioproducts. Currently much work has been done to generate PHA (poly(3-hydroxyalkanoate)) molecules using recombinant bacteria. Work has been done to isolate and identify genes to produce PHAs for use in medical devices or for use as a biodegradable plastic in consumer products. There is also a body of work describing the use of transgenic plants to produce PHAs for use in biodegradable plastics. Moreover, several studies demonstrating PHA production by mixed microbial communities fed a variety of complex feedstocks under a variety of cultivation conditions were performed and resulted in yields that showed economic feasibility as a starting precursor compound for commercial chemical production. The presented invention relates to the use of mixed microbial communities to produce several related bioproducts that are derived from PHA molecules.

Current efforts in the biorefinery sector are being focused on generating multiple products such as bio-based products and bioenergy from single renewable feedstocks. The ability to produce multiple products from one biorefinery allows for a more flexible revenue stream and limits risk for investment by rapidly changing energy prices. Waste water treatment plants (WWTPs) currently use biogas production as the primary source of energy production. Historically digested biosolids produced at WWTPs have been trucked to landfills to be used as cover, but more recently municipalities have found other uses such as state forest land fertilizer as well as selling to commercial ventures for residential fertilizer utilization.

This process would allow for the production of high-value specialty chemicals in conjunction with lower value fuel compounds which would overall improve the economics of the biorefinery unit.

### BRIEF DESCRIPTION OF THE INVENTION

This present invention relates to the field of renewable biorefinery specialty chemicals. Current efforts to produce chemicals from renewable sources have been limited in their ability to significantly change the landscape of chemical production because of high costs and lack of renewable feed streams. This invention in particular relates to a family of molecules derived from conditions using waste streams as a feed source. These molecules are derived from naturally occurring chemical storage compounds (poly(3-hydroxyalkanoate) (PHA)) found in native bacteria. PHA molecules are found in many feedstocks where carbon is in excess. These feedstocks include, but are not limited to, agricultural residual biomass (e.g., corn stover, and rice straw), distiller's grains, food production by-products, fuel production biomass and WWTP biosolids. Currently much work with PHA compounds requires expensive extraction of the PHA compounds from biomass prior to further processing. This invention relates to the in-situ production of alkoxyalkanoate alcohol esters, derived from PHA compounds, which range from 4 to 21 carbons in length for use as a specialty chemical. In one aspect this invention relates to the use of ethyl 3-ethoxybutyrate (EEB) from renewable sources as a chemical solvent that could potentially replace ethyl 3-ethoxypropionate (EEP). Currently EEP has several solvent industrial applications including, high solids coatings, electrostatically spayed coatings, conventional enamels, and acrylic polymerization. Another aspect of this invention relates to the production of ethyl crotonate (EC) for use as an organic intermediate for bulk chemical production or as a plasticizer for acrylic resins.

Previously, work has been done to describe the production of alkoxyalkanoate alcohol esters for use as a biofuel. The process for production of EEB and EC is the same as described in the non-provisional patent filed on Nov. 2, 2009 (U.S. Pat. No. 8,377,151) and is summarized below.

A feed stream containing biomass with accumulated poly(3-hydroxyalkanoate) molecules is dehydrated. The resulting biomass is reacted in-situ with an alcohol in either an acid catalyzed unit operation or base catalyzed reaction unit operation followed by an esterification unit operation. The resulting products, including alkoxy esters and alkenoic esters such as EEB and EC, are separated. It is also possible in a subsequent reaction to convert EC into EEB under caustic conditions. This process provides a final, high quality renewable chemical product.

### DETAILED DESCRIPTION OF THE INVENTION

The specialty chemicals as described can be produced using much existing infrastructure at a WWTP. Influent entering a WWTP will typically undergo a cycling of anaerobic and aerobic digestion aimed at reducing chemical oxygen demand (COD) and biological oxygen demand (BOD). During this cycling PHA accumulation may occur in bacteria due to nutrient limitation and excess carbon. Typical WWTPs are capable of producing PHAs up to, but not limited to, 4 g PHA per 100 g wet slurry. This PHA stream may contain monomeric, oligomeric, and polymer PHA compounds. In most instances the slurry must then be dehydrated before further processing can occur.

Dehydration can be done using either solvent extraction, physical compression or by heating. Once the hydration content is reduced to less than 5% water w/w the slurry can be further processed.

In one aspect after dehydration the resulting biomass is then resuspended in an excess alcohol solution comprising ethanol at a ratio of 3.67 kg of ethanol for every kilogram of PHA.

In another aspect after dehydration the resulting biomass is then resuspended in an excess alcohol solution comprising alcohol from a selection consisting of methanol, propanol, and butanol, pentanol, hexanol, heptanol, or octanol at a ratio of 6 parts alcohol for each part PHA.

In another aspect after dehydration the resulting biomass is then resuspended in an excess alcohol solution comprising ethanol at a molar ratio of less than 6 parts ethanol for each part PHA.

In another aspect after dehydration the resulting biomass stream is resuspended in an excess alcohol solution containing denaturants such as ketones or light petroleum distillates.

In one aspect sulfuric acid can be added to the alcohol solution in the ratio of 0.65 g of sulfuric acid for each kilogram of PHA. The entire slurry is then heated to about 140.degree. C. and the reaction is allowed to proceed for about 2 hours. The resulting products contain ethyl 3-ethoxyalkanoates that can then be further purified to be used as a specialty chemical.

In another aspect sulfuric acid can be added to the alcohol solution in the ratio of 0.65 g of sulfuric acid for each kilogram of PHA. The entire slurry is then heated to about 140.degree. C. and the reaction is allowed to proceed for about 2 hours. The resulting products contain ethyl crotonate that can then be further purified to be used as a specialty chemical.

In another aspect sulfuric acid can be added to the alcohol solution in the ratio of 0.65 g of sulfuric acid for each kilogram of PHA. The entire slurry is then heated to about 140.degree. C. and the reaction is allowed to proceed for about 2 hours. The resulting products contain alkoxy esters that can then be further purified to be used as a specialty chemical.

In another aspect sulfuric acid can be added to an ethanol slurry containing PHA in the ratio of 0.65 g of sulfuric acid for each kilogram of PHA. The entire slurry can then be heated to between 60.degree. C. and 160.degree. C. for up to 240 mins. The resulting products contain ethyl 3-ethoxyalkanoates that can then be further purified to be used as a specialty chemical.

In another aspect sulfuric acid can be added to an ethanol slurry containing PHA in the ratio of 0.65 g of sulfuric acid for each kilogram of PHA. The entire slurry can then be heated to between 60.degree. C. and 160.degree. C. for up to 240 mins. The resulting products contain ethyl crotonate that can then be further purified to be used as a specialty chemical.

In another aspect sulfuric acid can be added to an ethanol slurry containing PHA in the ratio of 0.65 g of sulfuric acid for each kilogram of PHA. The entire slurry can then be heated to between 60.degree. C. and 160.degree. C. for up to 240 mins. The resulting products contain alkoxy esters that can then be further purified to be used as a specialty chemical.

In another aspect sulfuric acid can be added to an ethanol slurry containing PHA in the ratio of 0.65 g of sulfuric acid for each kilogram of PHA. The entire slurry can then be heated using microwave radiation to between 60.degree. C. and 160.degree. C. for between 1 minute and 120 minutes. The resulting products contain ethyl 3-ethoxyalkanoates that can then be further purified to be used as a specialty chemical.

In another aspect sulfuric acid can be added to an ethanol slurry containing PHA in the ratio of 0.65 g of sulfuric acid for each kilogram of PHA. The entire slurry can then be heated using microwave radiation to between 60.degree. C. and 160.degree. C. for between 1 minute and 120 minutes. The resulting products contain ethyl crotonate that can then be further purified to be used as a specialty chemical.

In another aspect sulfuric acid can be added to an ethanol slurry containing PHA in the ratio of 0.65 g of sulfuric acid for each kilogram of PHA. The entire slurry can then be heated using microwave radiation to between 60.degree. C. and 160.degree. C. for between 1 minute and 120 minutes. The resulting products contain alkoxy esters that can then be further purified to be used as a specialty chemical.

In another aspect one in-situ reaction of PHA with an alcohol solution results in a product stream containing ethyl crotonate. The ethyl crotonate is then separated from other products by means of solvent extraction and distillation. The ethyl crotonate is then heated to between 60.degree. C. and 160.degree. C. in an alkaline solution with ethanol at a ratio of 6 parts ethanol to 1 part ethyl crotonate for up to 240 minutes. The reaction product contains ethyl 3-ethoxyalkanoates that can then be further purified to be used as a specialty chemical.

In one aspect the first step of purification is done by the addition of a hexane solution to separate the specialty chemical mixture from the residual biosolids. The chemical products will partition to the hexane phase.

In one aspect after extraction into the hexane solution a distillation column can be used to separate the chemical products from the hexane and alcohol. The hexane and alcohol solutions can then be separated using a distillation column and recycled for reuse in another round of esterifications/transesterification and extractions. The resultant chemical product stream can then be further purified using another distillation column to remove any residual alcohol or hexane.

Further purification of the chemical mixture may be necessary depending on the purity required as a reagent for subsequent reactions.

In one aspect the chemical product stream contains a mixture of specialty chemicals including ethyl crotonate, ethyl 3-hydroxybutyrate, and ethyl 3-ethoxybutyrate.

In one aspect the chemical product stream contains a mixture of specialty chemicals including various alkoxy esters.

We claim:

1. A method of producing renewable specialty chemicals comprising the following steps, dehydrating a stream containing biomass that contains PHA molecules, suspending the dehydrated PHA in an alcohol solution containing a catalyst to form a slurry, heating the slurry containing PHA until an in-situ reaction occurs with the PHA thereby forming the chemical products, and separating the resulting products to obtain the specialty chemicals.

2. The method of claim 1 wherein the method of dehydration is selected from the group consisting of heating, solvent extraction, and compression.

3. The method of claim 1 wherein the stream being dehydrated contains PHA monomers, oligomers, and polymers.

4. The method of claim 1 wherein the PHA molecules comprise poly(3-hydroxybutyrate) (PHB).

5. The method of claim 1 wherein the PHA molecules comprise a copolymer of poly(3-hydroxy-butyrate and 3-hydroxyvalerate) (PHB/V)

6. The method of claim 1 wherein the PHA molecules comprise a mixture of PHB, PHBN, and medium-chain-length poly(3-hydroxyalkanoate) (mcl-PHA).

7. The method of claim 1 wherein the alcohol solution comprises short chain alcohols such as methanol, ethanol, propanol and butanol.

8. The method of claim 1 wherein the alcohol solution comprises long chain alcohols such as pentanol, hexanol, heptanol and octanol.

9. The method of claim 1 wherein the alcohol solution comprises ethanol.

10. The method of claim 1 wherein the catalyst contains sulfuric acid.

11. The method of claim 1 wherein the slurry containing PHA is heated to between 60.degree. C. and 160.degree. C.

12. The method of claim 1 wherein the step of heating the slurry containing PHA is done using microwave radiation.

13. The method of claim 1 wherein the slurry containing PHA is heated for between 1 minute and 240 minutes.

14. The method of claim 1 wherein the reaction occurring with the PHA slurry and the alcohol is an esterification.

15. The method of claim 1 wherein the reaction occurring with the PHA slurry and the alcohol is a transesterification.

16. The method of claim 1 wherein the step of separating the products is selected from the group consisting of distillation, centrifugation and solvent extraction.

17. The method of claim 1 wherein the resulting specialty chemicals product comprises ethyl ethoxyalkanoates.

18. The method of claim 1 wherein the resulting specialty chemicals product comprises ethyl crotonate.

19. The method of claim 1 wherein the resulting specialty chemicals product comprises ethyl 3-ethoxybutyrate.

20. The method of claim 1 wherein the source of the feedstock biomass is from waste water treatment plant biosolids.

21. The method of claim 1 wherein the source of feedstock biomass is from agricultural residue.

22. The method of claim 1 wherein the source of the feedstock biomass is from fuel production biomass.

23. The method of claim 1 wherein the source of the feedstock biomass is from biomass processing by-products.

24. The method of claim 1 wherein the resulting specialty chemicals product comprises ethyl crotonate and the ethyl crotonate is then further heated with ethanol under alkaline conditions to produce ethyl 3-ethoxybutyrate.
